(19) United States
(12) Patent Application Publication
(10) Pub. No.: US 2003/0181981 A1
(43) Pub. Date: Sep. 25, 2003

(54) Anterior lumbar interbody implant

(76) Inventor: Jean-Philippe Lemaire, Le Pre Loiseau (FR)

Correspondence Address: LADAS & PARRY, 224 SOUTH MICHIGAN AVENUE, SUITE 1200, CHICAGO, IL, 60604, US

(21) Appl. No.: 10/275,518

(22) PCT Filed: May 11, 2001
(86) PCT No.: PCT/FR01/01431

(30) Foreign Application Priority Data
May 11, 2000 (FR) 00/06023

(51) Int. Cl. A61F 2/44

(52) U.S. Cl. 623/17.11

(57) ABSTRACT

The invention relates to an intersomatic implant for insertion in the intervertebral space defined between two adjacent lumbar vertebrae, referred to as an overlying vertebra and an underlying vertebra, in order to reestablish the anatomical shape of the intervertebral space. According to the invention, the implant includes at least one spacer ramp (12a, 13a, 13b):

extending between the anterior and posterior transverse walls (4) so as to project relative to a transverse face (8) in order to determine relative spacing between the underlying and overlying vertebrae while the implant is being inserted into the intervertebral space; and

presenting a profile which is to be found circumscribed inside the intervertebral space in which the implant is to be placed.

The present invention relates to an intersomatic implant for insertion in the intervertebral space after excision of the disk, i.e. into the space between two adjacent vertebrae in order to restore an appropriate height between the vertebrae and to ensure that bone fusion takes place between said adjacent vertebrae.

More precisely, the invention relates to an intersomatic implant of the lumbar type for placing in an intervertebral space defined between two adjacent lumbar vertebrae after excision of the disk.

In the state of the art, it is known to insert an intersomatic implant in the intervertebral space defined between two adjacent lumbar vertebrae. Numerous embodiments of such implants have been proposed in the prior art. For example, document WO 97/23175 discloses a lumbar intersomatic implant in the form of a cage having two sagittal walls interconnected by an anterior transverse wall and by a posterior transverse wall. Between them, the walls define an open volume suitable for receiving a bone filler substance referred to as a bone graft, that is to come into contact with the spongy bone of each of the vertebral plates, serving to encourage bone fusion between the two vertebrae. That implant also has at least two walls projecting from its top and bottom faces and shaped to become inserted progressively in the spongy bone during insertion of the implant, and then to prevent it from moving.

It should be understood that a bone graft placed inside such an implant generally leaves portions that project relative to the transverse faces of the implant. Unfortunately, while the cage is being implanted in the intervertebral space, rubbing occurs between the implant and the vertebrae, leading to damage to those portions of the bone graft that are situated level with the transverse faces of the implant. As a result such a bone graft is not in contact with the spongy bone of the vertebral plates of the vertebrae after the cage has been implanted.

The invention seeks to remedy the above-specified drawbacks by proposing an intersomatic implant that is to be inserted in the intervertebral space defined between two adjacent lumbar vertebrae, and designed by means of a bone graft placed inside the implant to enable good bone continuity to be established between the spongy bone of the vertebral plates of the two adjacent lumbar vertebrae.

To achieve this object, the intersomatic implant is to be inserted in the intervertebral space defined between two adjacent lumbar vertebrae, referred to as an overlying vertebra and an underlying vertebra, in order to reestablish the anatomical shape of the intervertebral space, the implant being in the form of a cage having at least two sagittal walls interconnected at least by an anterior transverse wall and by a posterior transverse wall that are substantially parallel to a front plane, the walls defining between them an open volume for bone filler and presenting rims extending on one side to define a first transverse face and on the other side to define a second transverse face.

According to the invention, the implant includes at least one spacer ramp:

extending between the anterior and posterior transverse walls so as to project relative to a transverse face in order to determine relative spacing between the underlying and overlying vertebrae while the implant is being inserted into the intervertebral space; and

presenting a profile which is to be found circumscribed inside the intervertebral space in which the implant is to be placed.

Various other characteristics appear from the following description given with reference to the accompanying drawings which show particular embodiments of the invention as non-limiting examples.

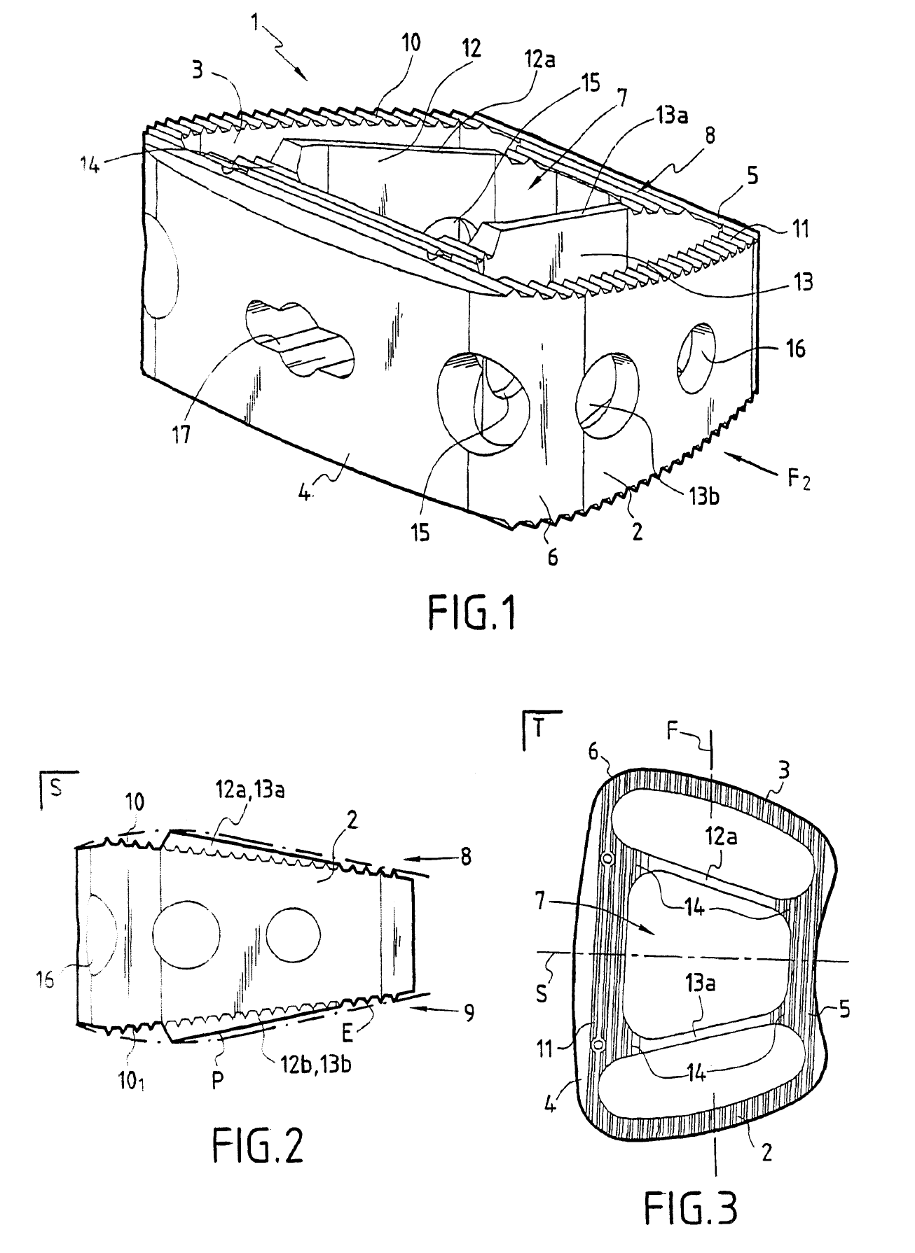

FIG. 1 is a perspective view of an embodiment of an implant in accordance with the invention.

FIG. 2 is a sagittal view of an implant seen substantially along arrow F₂ of FIG. 1.

FIG. 3 is a transverse view of the implant shown in FIGS. 1 and 2.

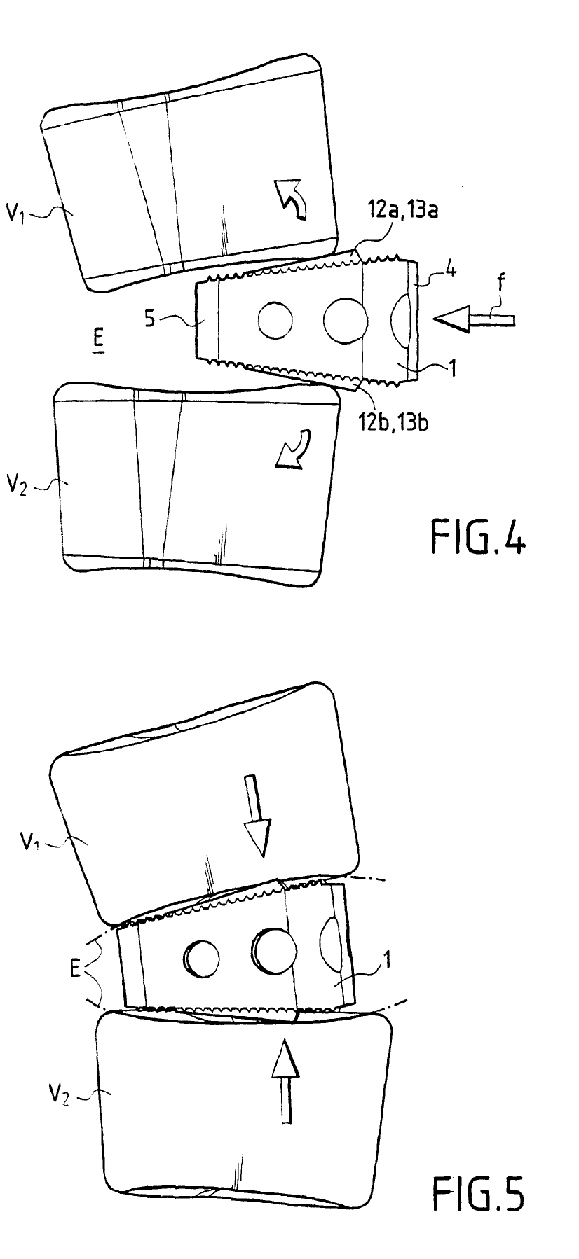

FIGS. 4 and 5 are sagittal views serving to explain the advantages of the invention, and showing the implant of the invention respectively in a position while it is being implanted, and in a final, implanted position.

As can be seen more precisely from FIGS. 1 to 3, the intersomatic implant of the invention is in the form of a cage 1 of generally rectangular shape for insertion in the intervertebral space after excision of the disk, i.e. the space defined between two adjacent lumbar vertebrae and referred to in the description below as the intervertebral space E. The cage 1 has a first sagittal wall 2 and a second sagittal wall 3 interconnected by an anterior transverse wall 4 and a posterior transverse wall 5. In the example shown, the sagittal walls 2 and 3 extend perpendicularly to a transverse plane T and slope relative to a plane S, referred to as a sagittal or antero-posterior plane that is perpendicular to the plane T. More precisely, the sagittal walls 2 and 3 converge going away from the anterior wall 4. The anterior and posterior transverse walls 4 and 5 extend substantially parallel to each other and to a front plane F perpendicular to the sagittal plane S.

The cage 1 preferably has connection fillets 6 formed between the sagittal walls 2 and 3 and the transverse walls 4 and 5 at their vertical outside faces so as to provide a cage 1 having corners that are rounded on the outside. As can be seen more clearly from FIG. 3, each sagittal wall 2 and 3, and also the anterior transverse wall 4, possesses convex curvature in the plane T perpendicular to the front plane F, whereas the posterior transverse wall 5 possesses concave curvature so as to leave the vertebral channel free.

On the inside, the cage 1 presents a volume 7 defined by the inside vertical faces of the walls 2 to 5, which volume is to be filled with a bone filler substance referred to as a bone graft and adapted to intersomatic fusion. This volume 7 opens out into a first transverse face 8, the top face in the example shown, and a second transverse face 9, the bottom face in the example shown. The walls 2 to 5 present rims 10 defining the top transverse face 8 and rims 10₁ defining the bottom transverse face 9.

The cage 1 has serrations 11 formed in the rims 10 and 10₁ of the walls 2 to 5 so as to enable the cage to engage the overlying and underlying vertebrae. The serrations 11 extend parallel to one another and to the front plane F. In general, it should be understood that the top and bottom transverse faces 8 and 9 corresponding to the envelope containing the tips of the serrations 11.

In accordance with the invention, the cage 1 further includes at least one, and in the example shown four, ramps 12a, 12b, 13a, 13b each extending between the front and rear transverse walls 4 and 5 and adapted, designed, or shaped so as to serve to hold the vertebrae apart, as can be seen from the description below. Each spacer ramp 12a-13a and 12b-13b projects relative to an adjacent transverse face, respectively the face 8 or the face 9. Relative to the adjacent transverse face 8 or 9, each spacer ramp 12a, 12b, 13a, 13b is of a profile which increases progressively going towards the anterior transverse wall 4. In the example shown, the spacer ramps 12a, 12b, 13a, 13b extend perpendicularly to the transverse plane T, sloping relative to the sagittal plane S. More precisely, the spacer ramps 12a and 13a, and the spacer ramps 12b and 13b converge going away from the anterior wall 4, and they are symmetrical about the sagittal plane S.

The spacer ramps 12a, 12b and 13a, 13b project from the sagittal faces 8 and 9 and converge towards each other towards the posterior transverse wall 5 such that the cage 1 is frustoconical in section in the sagittal plane S, thereby making it possible to define an angle for reestablishing curvature of the spine. Selecting heights for the anterior and posterior transverse walls 4 and 5 makes it possible to provide a range of cages suitable for reestablishing different angles of curvature.

In an advantageous embodiment, each spacer ramp 12a, 12b, 13a, 13b is connected to each transverse wall 4, 5 via a connection zone 14 extending the transverse wall at the same level as the corresponding portion of the sagittal walls 2, 3. It should be understood that the profiles of these connection zones 14 match the profiles of the correspondingly situated sagittal walls 2, 3. These connection zones 14 present edges that are likewise provided with serrations 11. It should thus be considered that the spacer ramps 12a, 12b, 13a, 13b are shorter in length than the sagittal walls 2, 3.

In another advantageous embodiment, the ramps 12a, 12b, 13a, 13b present a profile P which is circumscribed inside the intervertebral space E, as represented by continuous lines in FIG. 2 and in which the implant is to be placed. It should be understood that these spacer ramps which project from the transverse faces 8 and 9 of the implant are dimensioned so as to remain in the intervertebral space E and avoid penetrating into the underlying and overlying vertebrae.

FIGS. 4 and 5 show more explicitly the functions of the spacer ramps of the cage 1 of the invention.

The cage 1 is intended to restore the intervertebral space E between two lumbar vertebrae V₁ and V₂ that are shown diagrammatically. As can be seen in FIG. 4, while the implant is being put into place along a path that is initially anterior, the cage 1 is moved in an insertion direction f lying in the sagittal plane S. While the cage 1 is being inserted into the intervertebral space E with the posterior transverse wall 5 situated downstream in the insertion direction, the spacer ramps 12a, 12b, 13a, 13b serve to move or space apart the overlying and underlying vertebrae V₁ and V₂. The presence of such spacer ramps makes it possible to avoid scraping the overlying and underlying vertebrae V₁ and V₂, thus making it possible to ensure that the overlying and underlying vertebrae V₁ and V₂ do not scrape the bone graft placed in the volume 7 of the cage. Given the profile of the spacer ramps 12a, 12b, 13a, 13b which increases going away from the posterior transverse wall 5 towards the anterior transverse wall 4, the two vertebrae V₁ and V₂ can be spaced apart progressively. Once the cage 1 is in its final position in the intervertebral space E (FIG. 5), it can be seen that the spacer ramps 12a, 12b, 13a, 13b remain confined inside the intervertebral space E defined between the overlying and underlying vertebrae. The cage 1 is held between the overlying and underlying vertebrae by the rims of the walls 2 to 5 which bear against the vertebral space. Given that the bone graft placed inside the cage 1 is not expelled in full or in part from the volume 7 while the implant is being put into place, good contact can be obtained between the bone graft and the spongy bone of the vertebral plates of the vertebrae V₁ and V₂.

In the embodiment shown, the cage 1 has four spacer ramps 12a, 12b, 13a, 13b. It should be understood that the spacer ramps 12a and 12b together define a first intermediate wall 12 extending in the same plane that is inclined relative to the sagittal plane S. Likewise, the spacer ramps 13a and 13b together define a second intermediate wall 13 extending in the same plane that is inclined relative to the sagittal plane S. These intermediate walls 12 and 13 preferably extend symmetrically about the sagittal plane S of the implant.

The intermediate walls 12 and 13 thus subdivide the filler volume 7 into three individual volumes. These intermediate walls 12 and 13 preferably have holes 15 allowing communication to be established between the various individual walls defined between them. The sagittal walls 2 and 3 and the anterior transverse wall 4 preferably likewise have through holes 16 encouraging. vascularization of the bone graft. It should be observed that the anterior transverse wall 4 has a slot 17 for receiving a tool that enables the bone graft placed inside the volume 7 to be compacted.

In the example shown, the cage 1 has four spacer ramps 12a, 12b, 13a, 13b. Naturally, it could be envisaged to make a cage 1 having a single spacer ramp. It could also be envisaged to make a cage having two spacer ramps lying in the same plane.

The invention is not limited to the embodiments described and shown, since various modifications can be made thereto without going beyond the ambit of the invention.

1/ An intersomatic implant for insertion in the intervertebral space (E) defined between two adjacent lumbar vertebrae, referred to as an overlying vertebra (V₁) and an underlying vertebra (V₂), in order to reestablish the anatomical shape of the intervertebral space, the implant being in the form of a cage having at least two sagittal walls (2, 3) interconnected at least by an anterior transverse wall (4) and by a posterior transverse wall (5) that are substantially parallel to a front plane, the walls defining between them an open volume (7) for bone filler and presenting rims extending on one side to define a first transverse face (8) and on the other side to define a second transverse face (9),
the implant being characterized in that it includes at least one spacer ramp (12a, 12b, 13a, 13b):
extending between the anterior and posterior transverse walls (4, 5) so as to project relative to a transverse face (8, 9) in order to determine relative spacing between the underlying and overlying vertebrae while the implant is being inserted into the intervertebral space; and
presenting a profile (P) which is to be found circumscribed inside the intervertebral space (E) in which the implant is to be placed.

2/ An implant according to claim 1, characterized in that it has two spacer ramps (12a, 12b) defining a first intermediate wall (12) extending in a single plane, each spacer ramp projecting relative to a respective one of the transverse faces (8, 9).

3/ An implant according to claim 2, characterized in that it has a second intermediate wall (13) extending symmetrically to the other intermediate wall, said second intermediate wall (13) being provided with two spacer ramps (13a, 13b) each projecting from a respective one of the transverse faces (8, 9).

4/ An implant according to any one of claims 1 to 3, characterized in that each spacer ramp (12a, 12b, 13a, 13b) possesses a profile relative to the adjacent transverse face which increases progressively going towards the anterior transverse wall (4).

5/ An implant according to any one of claims 1 to 4, characterized in that each spacer ramp (12a, 12b, 13a, 13b) is connected to a transverse wall (4, 5) by a connection zone (14) extending said transverse wall level with the sagittal walls so that each spacer ramp possesses a length that is shorter than the length of a sagittal wall (2, 3).

6/ An implant according to any one of claims 1 to 3, characterized in that the sagittal walls (2, 3) and/or the intermediate walls (12, 13) have through holes (15, 16).

7/ An implant according to claims 1 and 5, characterized in that the rims of the sagittal and transverse walls (2, 3; 4, 5) and the rims of the connection zone (14) include serrations (11).

8/ An implant according to claim 2, characterized in that the spacer ramps (12a, 12b, 13a, 13b) project relative to the sagittal faces (8, 9) which converge towards each other towards the posterior transverse wall (5) so that the cage is of frustoconical section in the sagittal plane (S) in order to define an angle for reestablishing lordosis.

9/ An implant according to claim 1, characterized in that the posterior transverse wall (5) possesses concave curvature so as to leave the vertebral channel free.

10/ An implant according to claim 1, characterized in that the anterior transverse wall (4) presents a slot (17) for passing a tool that serves to compact the bone filling that is to be placed inside the implant.
